SEQUENCE LISTING (4 sequences)

SEQ ID NO: 1 - RNA (DNA from 25S rRNA gene), 318 nt; single-stranded, linear
  hypothetical: NO
  anti-sense: NO
  fragment type: genomic DNA
gacccgtcttgaaacacggaccaaggagtctaacgtctatgcaagtgtttgggtgtgaaacccgtacgcgtaatgaaagtgaacgtaggttggggccccttctggggtgcacaatcgaccgatcctgatgttctcggaaggatttgagtaagagcatagctgttaggacccgaaagatggtgaactatgcctgaatagggtgaagccagaggaaactctggtggaggctcgtagcggttctgacgtgcaaatcgatcgtcgaatttgggtataggggcgaaagactaatcgaaccatctagtagctggttccttccga

SEQ ID NO: 2 - RNA (DNA from 25S rRNA gene), 318 nt; single-stranded, linear; identical to SEQ ID NO: 1
  hypothetical: NO
  anti-sense: NO
  fragment type: genomic DNA

SEQ ID NO: 3 - RNA (DNA from 25S rRNA gene), 318 nt; single-stranded, linear
  hypothetical: NO
  anti-sense: NO
  fragment type: genomic DNA
gacccgtcttgaaacacggaccaaggagtctaacgtctatgcgagtgtttgggtgtaaaacccatacgcgtaatgaaagtgaacgtaggttggggcctcgcaagaggtgcacaatcgaccgatcctgatgtcttcggatggatttgagtaagagcatagctgttgggacccgaaagatggtgaactatgcctgaatagggtgaagccagaggaaactctggtggaggctcgtagcggttctgacgtgcaaatcgatcgtcgaatttgggtataggggcgaaagactaatcgaaccatctagtagctggttccttccga

SEQ ID NO: 4 - RNA (DNA from 25S rRNA gene), 318 nt; single-stranded, linear; identical to SEQ ID NO: 3
  hypothetical: NO
  anti-sense: NO
  fragment type: genomic DNA

(19) United States
(12) Patent Application Publication
(10) Pub. No.: US 2003/0157506 A1
(43) Pub. Date: Aug. 21, 2003

(54) Interferon-alpha induced genes

(76) Inventors: Jean-Francois Meritet, Paris (FR); Michel Dron, Bourg la Reine (FR); Michael Gerard Tovey, Paris (FR)

Correspondence Address: ALSTON & BIRD LLP, BANK OF AMERICA PLAZA, 101 SOUTH TRYON STREET, SUITE 4000, CHARLOTTE, NC, 28280-4000, US

(21) Appl. No.: 10/203,145

(22) PCT Filed: Feb. 9, 2001
(86) PCT No.: PCT/GB01/00578

(30) Foreign Application Priority Data
Feb. 11, 2000 (GB) 00032303.7
Feb. 11, 2000 (GB) 0003204.5
Feb. 11, 2000 (GB) 0003205.2
Feb. 11, 2000 (GB) 0003206.0
Feb. 11, 2000 (GB) 0003207.8
Feb. 11, 2000 (GB) 0003208.6
Feb. 11, 2000 (GB) 0003210.2
Feb. 11, 2000 (GB) 0003212.8
Feb. 11, 2000 (GB) 0003213.6
Feb. 11, 2000 (GB) 0003215.1
Feb. 11, 2000 (GB) 0003216.9
Feb. 11, 2000 (GB) 0003219.3
Feb. 11, 2000 (GB) 0003220.1
Feb. 11, 2000 (GB) 0003221.9
Feb. 11, 2000 (GB) 0003222.7
Feb. 17, 2000 (GB) 0003768.9

(51) Int. Cl. C12Q 1/68; G01N 33/53

(52) U.S. Cl. 435/6; 435/7.1

(57) ABSTRACT

The present disclosure relates to identification of previously known genes as being genes upregulated by interferon-α administration, in particular the human genes corresponding to the cDNA sequence in GenBank designated g4758303, g5453897, g4505186, g2366751, g33917, g4504962, g3978516, g5924396, g4505656, g1504007, g3702446, g4001802, g292289, g4557226, g4507646 and g4507170. Determination of expression products of these genes is proposed as having utility in predicting responsiveness to treatment with interferon-α and other interferons which act at the Type 1 interferon receptor.

### FIELD OF THE INVENTION

The present invention relates to identification of previously known genes as genes upregulated by interferon-α (IFN-α) administration. Detection of expression products of these genes is thus now proposed as a means for predicting responsiveness to IFN-α and other interferons which act at the Type 1 interferon receptor.

### BACKGROUND OF THE INVENTION

IFN-α is widely used for the treatment of a number of disorders. Disorders which may be treated using IFN-α include neoplastic diseases such as leukemia, lymphomas, and solid tumours, AIDS-related Kaposi's sarcoma and viral infections such as chronic hepatitis. IFN-α has also been proposed for administration via the oromucosal route for the treatment of autoimmune, mycobacterial, neurodegenerative, parasitic and viral disease. In particular, IFN-α has been proposed, for example, for the treatment of multiple sclerosis, leprosy, tuberculosis, encephalitis, malaria, cervical cancer, genital herpes, hepatitis B and C, HIV, HPV and HSV-1 and 2. It has also been suggested for the treatment of arthritis, lupus and diabetes. Neoplastic diseases such as multiple myeloma, hairy cell leukemia, chronic myelogenous leukemia, low grade lymphoma, cutaneous T-cell lymphoma, carcinoid tumours, cervical cancer, sarcomas including Kaposi's sarcoma, kidney tumours, carcinomas including renal cell carcinoma, hepatic cellular carcinoma, nasopharyngeal carcinoma, haematological malignancies, colorectal cancer, glioblastoma, laryngeal papillomas, lung cancer, colon cancer, malignant melanoma and brain tumours are also suggested as being treatable by administration of IFN-α via the oromucosal route, i.e. the oral route or the nasal route.

IFN-α is a member of the Type 1 interferon family, which exert their characteristic biological activities through interaction with the Type 1 interferon receptor. Other Type 1 interferons include IFN-β, IFN-ω and IFN-τ.

Unfortunately, not all potential patients for treatment with a Type 1 interferon such as interferon-α, particularly, for example, patients suffering from chronic viral hepatitis, neoplastic disease and relapsing remitting multiple sclerosis, respond favourably to Type 1 interferon therapy and only a fraction of those who do respond exhibit long-term benefit. The inability of the physician to confidently predict the therapeutic outcome of Type 1 interferon treatment raises serious concerns as to the cost-benefit ratio of such treatment, not only in terms of wastage of an expensive biopharmaceutical and lost time in therapy, but also in terms of the serious side effects to which the patient is exposed. Furthermore, abnormal production of IFN-α has been shown to be associated with a number of autoimmune diseases. For these reasons, there is much interest in identifying Type 1 interferon responsive genes since Type 1 interferons exert their therapeutic action by modulating the expression of a number of genes. Indeed, it is the specific pattern of gene expression induced by Type 1 interferon treatment that determines whether a patient will respond favourably or not to the treatment.

### SUMMARY OF THE INVENTION

It has now been found that the human genes corresponding to the cDNA sequences in GenBank assigned accession nos. g4758303, g5453897, g4505186, g2366751, g33917, g4504962, g3978516, g5924396, g4505656, g1504007, g3702446, g4001802, g292289, g4557226, g4507646 and g4507170 correspond to mouse genes upregulated by administration of IFN-α by an oromucosal route or intravenously.

The human gene corresponding to the cDNA sequence in GenBank assigned accession no. g4758303 was previously noted in GenBank as encoding a protein disulphide isomerase-related protein (ERP-70) but was not previously recognised as being of interest in relation to Type 1 interferon administration. It is now also designated an IFN-α upregulated gene.

The human gene corresponding to the cDNA sequence in GenBank assigned accession no g5453897 was previously noted in GenBank as encoding a protein termed peptidyl-prolyl cis/trans isomerase NIMA-interacting 1 (PIN-1) but was not previously recognised as being of interest in relation to Type 1 interferon administration. It is now also designated an IFN-α upregulated gene.

The human gene corresponding to the cDNA sequence in GenBank assigned accession no g4505186 was previously noted in GenBank as encoding a monokine induced by gamma interferon (MIG) but was not previously recognised as being of interest in relation to Type 1 interferon administration. It is now also designated an IFN-α upregulated gene.

The human gene corresponding to the cDNA sequence in GenBank assigned accession no g2366751 was previously noted in GenBank as encoding a lysyl tRNA synthetase (LTS) but was not previously recognised as being of interest in relation to Type 1 interferon administration. It is now also designated an IFN-α upregulated gene.

The human gene corresponding to the cDNA sequence in GenBank assigned accession no g33917 was previously noted in GenBank as encoding a gamma-interferon inducible early response gene (IP-10) with homology to platelet proteins but was not previously recognised as being of interest in relation to Type 1 interferon administration. It is now also designated an IFN-α upregulated gene.

The human gene corresponding to the cDNA sequence in GenBank assigned accession no g4504962 was previously noted in GenBank as encoding Lipocalin I but was not previously recognised as being of interest in relation to Type 1 interferon administration. It is now also designated an IFN-α upregulated gene.

The human gene corresponding to the cDNA sequence in GenBank assigned accession no g3978516 was previously noted in GenBank as encoding SEC 63 but was not previously recognised as being of interest in relation to Type 1 interferon administration. It is now also designated an IFN-α upregulated gene.

The human gene corresponding to the cDNA sequence in GenBank assigned accession no g5924396 was previously noted in GenBank as encoding surfeit 6 but was not previously recognised as being of interest in relation to Type 1 interferon administration. It is now also designated an IFN-α upregulated gene.

The human gene corresponding to the cDNA sequence in GenBank assigned accession no g4505656 was previously noted in GenBank as encoding a cGMP-stimulated phosphodiesterase 2A (PDE2A) but was not previously recognised as being of interest in relation to Type 1 interferon administration. It is now also designated an IFN-α upregulated gene.

The human gene corresponding to the cDNA sequence in GenBank assigned accession no g1504007 was previously noted in GenBank as encoding KIAA0212 but was not previously recognised as being of interest in relation to Type 1 interferon administration. It is now also designated an IFN-α upregulated gene.

The human gene corresponding to the cDNA sequence in GenBank assigned accession no g3702446 was previously noted in GenBank as encoding a phosphatidylinositol 4-kinase (NPIK-B) but was not previously recognised as being of interest in relation to Type 1 interferon administration. It is now also designated an IFN-α upregulated gene.

The human gene corresponding to the cDNA sequence in GenBank assigned accession no g4001802 was previously noted in GenBank as encoding BAF53a but was not previously recognised as being of interest in relation to Type 1 interferon administration. It is now also designated an IFN-α upregulated gene.

The human gene corresponding to the cDNA sequence in GenBank assigned accession no g292289 was previously noted in GenBank as encoding a MADS/MEF2-family transcription factor (MEF2C) but was not previously recognised as being of interest in relation to Type 1 interferon administration. It is now also designated an IEN-α upregulated gene.

The human gene corresponding to the cDNA sequence in GenBank assigned accession no g4557226 was previously noted in GenBank as encoding an arylacetamide deacetylase (AADAC) but was not previously recognised as being of interest in relation to Type 1 interferon administration. It is now also designated an IFN-α upregulated gene.

The human gene corresponding to the cDNA sequence in GenBank assigned accession no g4507646 was previously noted in GenBank as encoding α tropomyosin 1 (TPM1) but was not previously recognised as being of interest in relation to Type 1 interferon administration. It is now also designated an IFN-α upregulated gene.

The human gene corresponding to the cDNA sequence in GenBank assigned accession no g4507170 was previously noted in GenBank as encoding secreted protein that is acidic and rich in cysteine (SPARC) but was not previously recognised as being of interest in relation to Type 1 interferon administration. It is now also designated an IFN-α upregulated gene.

Determination of the level of one or more of ERP-70, PIN-1, MIG, LTS, IP-10, Lipocalin 1, SEC 63, surfeit 6, PDE2A, KIAA0212, NPIK-B, BAF53a, MEF2C, AADAC, TPM1 or SPARC protein or a naturally-occurring variant thereof, or the corresponding mRNA, in cell samples of Type 1 interferon-treated patients, e.g. patients treated with IFN-α, e.g. such as by the oromucosal route or intravenously, may thus be used to predict responsiveness to such treatment. It has additionally been found that alternatively and more preferably, such responsiveness may be judged, for example, by treating a sample of human peripheral blood mononuclear cells in vitro with a Type 1 interferon and looking for upregulation or downregulation of expression products, preferably mRNA, corresponding to the same gene or genes.

### BRIEF DESCRIPTION OF THE SEQUENCES

SEQ. ID. No. 1 is the sequence of the cDNA designated in Genbank as accession no.g4758303 with the corresponding encoded polypeptide sequence shown below.

SEQ. ID. No.2 is the amino acid sequence alone of ERP-70 corresponding to GenBank accession no. g4758304.

SEQ. ID. No.3 is the sequence of the cDNA designated in Genbank as accession no.g5453897 with the corresponding encoded polypeptide sequence shown below.

SEQ. ID. No.4 is the amino acid sequence alone of PIN-1 corresponding to GenBank accession no. g5453898.

SEQ. ID. No.5 is the sequence of the cDNA designated in Genbank as accession no.g4505186 with the corresponding encoded polypeptide sequence shown below.

SEQ. ID. No.6 is the amino acid sequence alone of MIG corresponding to GenBank accession no. g4505187.

SEQ. ID. No.7 is the sequence of the cDNA designated in Genbank as accession no.g2366751 with the corresponding encoded polypeptide sequence shown below.

SEQ. ID. No.8 is the amino acid sequence alone of LTS corresponding to GenBank accession no. g2366752.

SEQ. ID. No.9 is the sequence of the cDNA designated in Genbank as accession no.g33917 with the corresponding encoded polypeptide sequence shown below.

SEQ. ID. No.10 is the amino acid sequence alone of IP-10 corresponding to GenBank accession no. g33918.

SEQ. ID. No.11 is the sequence of the cDNA designated in Genbank as accession no.g4504962 with the corresponding encoded polypeptide sequence shown below.

SEQ. ID. No.12 is the amino acid sequence alone of Lipocalin 1 corresponding to GenBank accession no. g4504963.

SEQ. ID. No.13 is the sequence of the cDNA designated in Genbank as accession no.g3978516 with the corresponding encoded polypeptide sequence shown below.

SEQ. ID. No.14 is the amino acid sequence alone of SEC 63 corresponding to GenBank accession no. g3978517.

SEQ. ID. No.15 is the sequence of the cDNA designated in Genbank as accession no.g5924396 with the corresponding encoded polypeptide sequence shown below.

SEQ. ID. No.16 is the amino acid sequence alone of surfeit 6 corresponding to GenBank accession no. g5924396.

SEQ. ID. No.17 is the sequence of the cDNA designated in Genbank as accession no.g4505656 with the corresponding encoded polypeptide sequence shown below.

SEQ. ID. No.18 is the amino acid sequence alone of PDE2A corresponding to GenBank accession no. g4505656.

SEQ. ID. No. 19 is the sequence of the cDNA designated in Genbank as accession no.g1504007 with the corresponding encoded polypeptide sequence shown below.

SEQ. ID. No.20 is the amino acid sequence alone of KIAA0212 corresponding to GenBank accession no. g1504008.

SEQ. ID. No.21 is the sequence of the cDNA designated in Genbank as accession no.g3702446 with the corresponding encoded polypeptide sequence shown below.

SEQ. ID. No.22 is the amino acid sequence alone of NPIK-B corresponding to GenBank accession no. g3702447.

SEQ. ID. No.23 is the sequence of the cDNA designated in Genbank as accession no.g4001802 with the corresponding encoded polypeptide sequence shown below.

SEQ. ID. No.24 is the amino acid sequence alone of BAF53a corresponding to GenBank accession no. g4001803.

SEQ. ID. No.25 is the sequence of the cDNA designated in Genbank as accession no.g292289 with the corresponding encoded polypeptide sequence shown below.

SEQ. ID. No.26 is the amino acid sequence alone of MEF2C corresponding to GenBank accession no. g292290.

SEQ. ID. No.27 is the sequence of the cDNA designated in Genbank as accession no.g4557226 with the corresponding encoded polypeptide sequence shown below.

SEQ. ID. No.28 is the amino acid sequence alone of AADAC corresponding to GenBank accession no. g4557226.

SEQ. ID. No.29 is the sequence of the cDNA designated in Genbank as accession no.g4507646 with the corresponding encoded polypeptide sequence shown below.

SEQ. ID. No.30 is the amino acid sequence alone of TPM1 corresponding to GenBank accession no. g4507647.

SEQ. ID. No.31 is the sequence of the cDNA designated in Genbank as accession no.g4507170 with the corresponding encoded polypeptide sequence shown below.

SEQ. ID. No.32 is the amino acid sequence alone of SPARC corresponding to GenBank accession no. g4507171.

### DETAILED DESCRIPTION

The present invention provides a method of predicting responsiveness of a patient to treatment with a Type 1 interferon, e.g. IFN-α treatment (such as IFN-α treatment by the oromucosal route or a parenteral route, for example, intravenously, subcutaneously or intramuscularly), which comprises determining the level of one or more of proteins selected from the proteins defined by the sequences set forth in SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30 or SEQ ID NO:32 and naturally-occurring variants thereof, e.g. allelic variants, or one or more of the corresponding mRNAs, in a cell sample from said patient, wherein said sample is obtained from said patient following administration of a Type 1 interferon, or is treated prior to said determining with a Type 1 interferon such as IFN-α in vitro. Such determining may be combined with determination of any other protein or mRNA whose expression is known to be affected in human cells by Type 1 interferon administration e.g. IFN-α administration.

Preferably, the Type 1 interferon for testing responsiveness will be the Type 1 interferon selected for treatment. It may be administered by the proposed treatment route and at the proposed treatment dose. Preferably, the subsequent sample analysed may be, for example, a blood sample or a sample of peripheral blood mononuclear cells (PBMCs) isolated from a blood sample.

More conveniently and preferably, a sample obtained from the patient comprising PBMCs isolated from blood may be treated in vitro with a Type 1 interferon, e.g. at a dosage range of about 1 to 10,000 IU/ml. Such treatment may be for a period of hours, e.g. about 7 to 8 hours. Preferred treatment conditions for such in vitro testing may be determined by testing PBMCs taken from normal donors with the same interferon and looking for upregulation of an appropriate expression product. Again, the Type 1 interferon employed will preferably be the Type 1 interferon proposed for treatment of the patient, e.g. recombinant IFN-α. PBMCs for such testing may be isolated in conventional manner from a blood sample using Ficoll-Hypaque density gradients. An example of a suitable protocol for such in vitro testing of Type 1 interferon responsiveness is provided in Example 18 below.

The sample, if appropriate after in vitro treatment with a Type 1 interferon, may be analysed for the level of one or more of ERP-70, PIN-1, MIG, LTS, IP-10, Lipocalin 1, SEC 63, surfeit 6, PDE2A, KIAA0212, NPIK-B, BAF53a, MEF2C, AADAC, TPM1 or SPARC protein or a naturally-occurring variant thereof. This may be done using an antibody or antibodies capable of specifically binding one or more of ERP-70, PIN-1, MIG, LTS, IP-10, Lipocalin 1, SEC 63, surfeit 6, PDE2A, KIAA0212, NPIK-B, BAF53a, MEF2C, AADAC, TPM1 or SPARC protein and naturally-occurning variants thereof, eg. allelic variants thereof. Preferably, however, the sample will be analysed for mRNA encoding ERP-70, PIN-1, MIG, LTS, IP-10, Lipocalin 1, SEC 63, surfeit 6, PDE2A, KIAA0212, NPIK-B, BAF53a, MEF2C, AADAC, TPM1 or SPARC protein or a naturally-occurring variant thereof Such mRNA analysis may employ any of the techniques known for detection of mRNAs, e.g. Northern blot detection or mRNA differential display. A variety of known nucleic acid amplification protocols may be employed to amplify any mRNA of interest present in the sample, or a portion thereof, prior to detection. The mRNA of interest, or a corresponding amplified nucleic acid, may be probed for using a nucleic acid probe attached to a solid support. Such a solid support may be a micro-array carrying probes to determine the level of further mRNAs or amplification products thereof corresponding to Type 1 interferon upregulated genes, e.g. such genes identified as upregulated in response to oromucosal or intravenous administration of IFN-α. Methods for constructing such micro-arrays (also referred to commonly as nucleic acid, probe or DNA chips) are well-known (see, for example, EP-B 0476014 and 0619321 of Affymax Technologies N.V. and Nature Genetics Supplement January 1999 entitled “The Chipping Forecast”). The following examples illustrate the invention:

### EXAMPLES

### Example 1

Previous experiments had shown that the application of 5 μl of crystal violet to each nostril of a normal adult mouse using a P20 Eppendorf micropipette resulted in an almost immediate distribution of the dye over the whole surface of the oropharyngeal cavity. Staining of the oropharyngeal cavity was still apparent some 30 minutes after application of the dye. These results were confirmed by using ¹²⁵I-labelled recombinant human IFN-α1-8 applied in the same manner. The same method of administration was employed to effect oromucosal administration in the studies which are described below.

Six week old, male DBA/2 mice were treated with either 100,000 IU of recombinant murine interferon α (IFN α) purchased from Life Technologies Inc, in phosphate buffered saline (PBS), 10 μg of recombinant human interleukin 15 (IL-15) purchased from Protein Institute Inc, PBS containing 100 μg/ml of bovine serum albumin (BSA), or left untreated. Eight hours later, the mice were sacrificed by cervical dislocation and the lymphoid tissue was removed surgically from the oropharyngeal cavity and snap frozen in liquid nitrogen and stored at −80° C. RNA was extracted from the lymphoid tissue by the method of Chomczynski and Sacchi (Anal. Biochem. (1987) 162, 156-159) and subjected to mRNA Differential Display Analysis (Lang, P. and Pardee, A. B., Science, 257, 967-971).

Differential Display Analysis

Differential display analysis was carried out using the “Message Clean” and “RNA image” kits of the GenHunter Corporation essentially as described by the manufacturer. Briefly, RNA was treated with RNase-free DNase, and 1 μg was reverse-transcribed in 100 μl of reaction buffer using either one or the other of the three one-base anchored oligo-(dT) primers A, C, or G. RNA was also reverse-transcribed using one or the other of the 9 two-base anchored oligo-(dT) primers AA, CC, GG, AC, CA, GA, AG, CG, GC. All the samples to be compared were reverse transcribed in the same experiment, separated into aliquots and frozen. The amplification was performed with only 1 μl of the reverse transcription sample in 10 μl of amplification mixture containing Taq DNA polymerase and α-³³P dATP (3,000 Ci/mmole). Eighty 5′ end (HAP) random sequence primers were used in combination with each of the (HT11) A, C, G, AA, CC, GG, AC, CA, GA, AG, CG or GC primers. Samples were then run on 7% denaturing polyacrylamide gels and exposed to authoradiography. Putative differentially expressed bands were cut out, reamplified according to the instructions of the supplier, and further used as probes to hybridise Northern blots of RNA extracted from the oropharyngeal cavity of IFN treated, IL-15 treated, and excipient treated animals.

Cloning and Sequencing

Re-amplified bands from the differential display screen were cloned in the Sfr 1 site of the pPCR-Script SK(+) plasmid (Stratagene), and cDNA amplified from the rapid amplification of cDNA ends were isolated by TA cloning in the pCR3 plasmid (Invitrogen). DNA was sequenced using an automatic di-deoxy sequencer (Perkin Ehner ABI PRISM 377).

Identification of Human cDNA

Differentially expressed murine 3′ sequences identified from the differential display screen were compared with random human expressed sequence tags (EST) present in the dbEST database of GenBank™ of the United States National Center for Biotechnology Information (NCBI). The sequences potentially related to the murine EST isolated from the differential display screen were combined in a contig and used to construct a human consensus sequence corresponding to a putative eDNA.

One such cDNA was found to correspond to GenBank cDNA sequence g4758303. The corresponding polypeptide sequence is GenBank sequence g4758304, which is noted in GenBank as corresponding to ERP-70.

Other mouse genes upregulated in lymphoid tissue in response to oromucosal administration of IFN-α as described above have also been found to be upregulated in the spleen of mice in response to intravenous administration of IFN-α. A similar result is anticipated in respect of the mouse gene corresponding to the human gene identified by Genbank cDNA accesssion no. g4758303 when intravenous administration of IFN-α is carried out as described in Example 17 below.

Furthermore, mRNAs corresponding to human gene analogues of mouse genes found to be upregulated in response to oromucosal and intravenous administration of IFN-α have been found to be enhanced in human peripheral blood mononuclear cells following treatment with IFN-α in vitro. The same result is anticipated for mRNA corresponding to the cDNA as set forth in SEQ. ID. No. 1 when human peripheral blood mononuclear cells are treated with IFN-α as described in Example 18 below.

### Example 2

Previous experiments had shown that the application of 5 μl of crystal violet to each nostril of a normal adult mouse using a P20 Eppendorf micropipette resulted in an almost immediate distribution of the dye over the whole surface of the oropharyngeal cavity. Staining of the oropharyngeal cavity was still apparent some 30 minutes after application of the dye. These results were confirmed by using ¹²⁵I-labelled recombinant human IFN-α1-8 applied in the same manner. The same method of administration was employed to effect oromucosal administration in the studies which are described below.

Six week old, male DBA/2 mice were treated with either 100,000 IU of recombinant murine interferon α (IFN α) purchased from Life Technologies Inc, in phosphate buffered saline (PBS), 10 μg of recombinant human interleukin 15 (IL-15) purchased from Protein Institute Inc, PBS containing 100 μg/ml of bovine serum albumin (BSA), or left untreated. Eight hours later, the mice were sacrificed by cervical dislocation and the lymphoid tissue was removed surgically from the oropharyngeal cavity and snap frozen in liquid nitrogen and stored at −80° C. RNA was extracted from the lymphoid tissue by the method of Chomczynski and Sacchi (Anal. Biochem. (1987) 162, 156-159) and subjected to mRNA Differential Display Analysis (Lang, P. and Pardee, A. B., Science, 257, 967-971).

Differential Display Analysis

Differential display analysis was carried out using the “Message Clean” and “RNA image” kits of the GenHunter Corporation essentially as described by the manufacturer. Briefly, RNA was treated with RNase-free DNase, and 1 μg was reverse-transcribed in 100 μl of reaction buffer using either one or the other of the three one-base anchored oligo-(dT) primers A, C, or G. RNA was also reverse-transcribed using one or the other of the 9 two-base anchored oligo-(dT) primers AA, CC, GG, AC, CA, GA, AG, CG, GC. All the samples to be compared were reverse transcribed in the same experiment, separated into aliquots and frozen. The amplification was performed with only 1 μl of the reverse transcription sample in 10 μl of amplification mixture containing Taq DNA polymerase and α-³³P dATP (3,000 Ci/mmole). Eighty 5′ end (HAP) random sequence primers were used in combination with each of the (HT11) A, C, G, AA, CC, GG, AC, CA, GA, AG, CG or GC primers. Samples were then run on 7% denaturing polyacrylamide gels and exposed to authoradiography. Putative differentially expressed bands were cut out, reamplified according to the instructions of the supplier, and further used as probes to hybridise Northern blots of RNA extracted from the oropharyngeal cavity of IFN treated, IL-15 treated, and excipient treated animals.

Cloning and Sequencing

Re-amplified bands from the differential display screen were cloned in the Sfr 1 site of the pPCR-Script SK(+) plasmid (Stratagene), and cDNA amplified from the rapid amplification of cDNA ends were isolated by TA cloning in the pCR3 plasmid (Invitrogen). DNA was sequenced using an automatic di-deoxy sequencer (Perkin Elmer ABI PRISM 377).

Identification of Human cDNA

Differentially expressed murine 3′ sequences identified from the differential display screen were compared with random human expressed sequence tags (EST) present in the dbEST database of GenBank™ of the United States National Center for Biotechnology Information (NCBI). The sequences potentially related to the murine EST isolated from the differential display screen were combined in a contig and used to construct a human consensus sequence corresponding to a putative cDNA.

One such cDNA was found to correspond to GenBank cDNA sequence g5453897. The corresponding polypeptide sequence is GenBank sequence g5453898, which is noted in GenBank as corresponding to PIN-1.

Other mouse genes upregulated in lymphoid tissue in response to oromucosal administration of IFN-α as described above have also been found to be upregulated in the spleen of mice in response to intravenous administration of IFN-α. A similar result is anticipated in respect of the mouse gene corresponding to the human gene identified by Genbank cDNA accesssion no. g5453897 when intravenous administration of IFN-α is carried out as described in Example 17 below.

Furthermore, mRNAs corresponding to human gene analogues of mouse genes found to be upregulated in response to oromucosal and intravenous administration of IFN-α have been found to be enhanced in human peripheral blood mononuclear cells following treatment with IFN-α in vitro. The same result is anticipated for mRNA corresponding to the cDNA as set forth in SEQ. ID. No. 3 when human peripheral blood mononuclear cells are treated with IFN-α as described in Example 18 below.

### Example 3

Previous experiments had shown that the application of 5 μl of crystal violet to each nostril of a normal adult mouse using a P20 Eppendorf micropipette resulted in an almost immediate distribution of the dye over the whole surface of the oropharyngeal cavity. Staining of the oropharyngeal cavity was still apparent some 30 minutes after application of the dye. These results were confirmed by using ¹²⁵I-labelled recombinant human IFN-α1-8 applied in the same manner. The same method of administration was employed to effect oromucosal administration in the studies which are described below.

Six week old, male DBA/2 mice were treated with either 100,000 IU of recombinant murine interferon α (IFN α) purchased from Life Technologies Inc, in phosphate buffered saline (PBS), 10 μg of recombinant human interleukin 15 (IL-15) purchased from Protein Institute Inc, PBS containing 100 μg/ml of bovine serum albumin (BSA), or left untreated. Eight hours later, the mice were sacrificed by cervical dislocation and the lymphoid tissue was removed surgically from the oropharyngeal cavity and snap frozen in liquid nitrogen and stored at −80° C. RNA was extracted from the lymphoid tissue by the method of Chomczynski and Sacchi (Anal. Biochem. (1987) 162, 156-159) and subjected to mRNA Differential Display Analysis (Lang, P. and Pardee, A. B., Science, 257, 967-971).

Differential Display Analysis

Differential display analysis was carried out using the “Message Clean” and “RNA image” kits of the GenHunter Corporation essentially as described by the manufacturer. Briefly, RNA was treated with RNase-free DNase, and 1 μg was reverse-transcribed in 100 μl of reaction buffer using either one or the other of the three one-base anchored oligo-(dT) primers A, C, or G. RNA was also reverse-transcribed using one or the other of the 9 two-base anchored oligo-(dT) primers AA, CC, GG, AC, CA, GA, AG, CG, GC. All the samples to be compared were reverse transcribed in the same experiment, separated into aliquots and frozen. The amplification was performed with only 1 μl of the reverse transcription sample in 10 μl of amplification mixture containing Taq DNA polymerase and α-³³P dATP (3,000 Ci/mmole). Eighty 5′ end (HAP) random sequence primers were used in combination with each of the (HT11) A, C, G, AA, CC, GG, AC, CA, GA, AG, CG or GC primers. Samples were then run on 7% denaturing polyacrylamide gels and exposed to authoradiography. Putative differentially expressed bands were cut out, reamplified according to the instructions of the supplier, and further used as probes to hybridise Northern blots of RNA extracted from the oropharyngeal cavity of IFN treated, IL-15 treated, and excipient treated animals.

Cloning and Sequencing

Re-amplified bands from the differential display screen were cloned in the Sfr 1 site of the pPCR-Script SK(+) plasmid (Stratagene), and cDNA amplified from the rapid amplification of cDNA ends were isolated by TA cloning in the pCR3 plasmid (Invitrogen). DNA was sequenced using an automatic di-deoxy sequencer (Perkin Elmer ABI PRISM 377).

Identification of Human cDNA

Differentially expressed murine 3′ sequences identified from the differential display screen were compared with random human expressed sequence tags (EST) present in the dbEST database of GenBank™ of the United States National Center for Biotechnology Information (NCBI). The sequences potentially related to the murine EST isolated from the differential display screen were combined in a contig and used to construct a human consensus sequence corresponding to a putative cDNA.

One such cDNA was found to correspond to GenBank cDNA sequence g4505186. The corresponding polypeptide sequence is GenBank sequence g4505187, which is noted in GenBank as corresponding to MIG.

Other mouse genes upregulated in lymphoid tissue in response to oromucosal administration of IFN-α as described above have also been found to be upregulated in the spleen of mice in response to intravenous administration of IFN-α. A similar result is anticipated in respect of the mouse gene corresponding to the human gene identified by Genbank cDNA accesssion no. g4505186 when intravenous administration of IFN-α is carried out as described in Example 17 below.

Furthermore, mRNAs corresponding to human gene analogues of mouse genes found to be upregulated in response to oromucosal and intravenous administration of IFN-α have been found to be enhanced in human peripheral blood mononuclear cells following treatment with IFN-α in vitro. The same result is anticipated for mRNA corresponding to the cDNA as set forth in SEQ. ID. No. 5 when human peripheral blood mononuclear cells are treated with IFN-α as described in Example 18 below.

### Example 4

Previous experiments had shown that the application of 5 μl of crystal violet to each nostril of a normal adult mouse using a P20 Eppendorf micropipette resulted in an almost immediate distribution of the dye over the whole surface of the oropharyngeal cavity. Staining of the oropharyngeal cavity was still apparent some 30 minutes after application of the dye. These results were confirmed by using ¹²⁵I-labelled recombinant human IFN-α1-8 applied in the same manner. The same method of administration was employed to effect oromucosal administration in the studies which are described below.

Six week old, male DBA/2 mice were treated with either 100,000 IU of recombinant murine interferon α (IFN α) purchased from Life Technologies Inc, in phosphate buffered saline (PBS), 10 μg of recombinant human interleukin 15 (IL-15) purchased from Protein Institute Inc, PBS containing 100 μg/ml of bovine serum albumin (BSA), or left untreated. Eight hours later, the mice were sacrificed by cervical dislocation and the lymphoid tissue was removed surgically from the oropharyngeal cavity and snap frozen in liquid nitrogen and stored at −80° C. RNA was extracted from the lymphoid tissue by the method of Chomczynski and Sacchi (Anal. Biochem. (1987) 162, 156-159) and subjected to mRNA Differential Display Analysis (Lang, P. and Pardee, A. B., Science, 257, 967-971).

Differential Display Analysis

Differential display analysis was carried out using the “Message Clean” and “RNA image” kits of the GenHunter Corporation essentially as described by the manufacturer. Briefly, RNA was treated with RNase-free DNase, and 1 μg was reverse-transcribed in 100 μl of reaction buffer using either one or the other of the three one-base anqhored oligo-(dT) primers A, C, or G. RNA was also reverse-transcribed using one or the other of the 9 two-base anchored oligo-(dT) primers AA, CC, GG, AC, CA, GA, AG, CG, GC. All the samples to be compared were reverse transcribed in the same experiment, separated into aliquots and frozen. The amplification was performed with only 1 μl of the reverse transcription sample in 10 μl of amplification mixture containing Taq DNA polymerase and α-³³P dATP (3,000 Ci/mmole). Eighty 5′ end (HAP) random sequence primers were used in combination with each of the (HT11) A, C, G, AA, CC, GG, AC, CA, GA, AG, CG or GC primers. Samples were then run on 7% denaturing polyacrylamide gels and exposed to authoradiography. Putative differentially expressed bands were cut out, reamplified according to the instructions of the supplier, and further used as probes to hybridise Northern blots of RNA extracted from the oropharyngeal cavity of IFN treated, IL-15 treated, and excipient treated animals.

Cloning and Sequencing

Re-amplified bands from the differential display screen were cloned in the Sfr 1 site of the pPCR-Script SK(+) plasmid (Stratagene), and cDNA amplified from the rapid amplification of cDNA ends were isolated by TA cloning in the pCR3 plasmid (Invitrogen). DNA was sequenced using an automatic di-deoxy sequencer (Perkin Elmer ABI PRISM 377).

Identification of Human cDNA

Differentially expressed murine 3′ sequences identified from the differential display screen were compared with random human expressed sequence tags (EST) present in the dbEST database of GenBank™ of the United States National Center for Biotechnology Information (NCBI). The sequences potentially related to the murine EST isolated from the differential display screen were combined in a contig and used to construct a human consensus sequence corresponding to a putative cDNA.

One such cDNA was found to correspond to GenBank cDNA sequence g2366751. The corresponding polypeptide sequence is GenBank sequence g2366752, which is noted in GenBank as corresponding to LTS.

Other mouse genes upregulated in lymphoid tissue in response to oromucosal administration of IFN-α as described above have also been found to be upregulated in the spleen of mice in response to intravenous administration of IFN-α. A similar result is anticipated in respect of the mouse gene corresponding to the human gene identified by Genbank cDNA accesssion no. g2366751 when intravenous administration of IFN-α is carried out as described in Example 17 below.

Furthermore, mRNAs corresponding to human gene analogues of mouse genes found to be upregulated in response to oromucosal and intravenous administration of IFN-α have been found to be enhanced in human peripheral blood mononuclear cells following treatment with IFN-α in vitro. The same result is anticipated for mRNA corresponding to the cDNA as set forth in SEQ. ID. No. 7 when human peripheral blood mononuclear cells are treated with IFN-α as described in Example 18 below.

### Example 5

Previous experiments had shown that the application of 5 μl of crystal violet to each nostril of a normal adult mouse using a P20 Eppendorf micropipette resulted in an almost immediate distribution of the dye over the whole surface of the oropharyngeal cavity. Staining of the oropharyngeal cavity was still apparent some 30 minutes after application of the dye. These results were confirmed by using ¹²⁵I-labelled recombinant human IFN-α1-8 applied in the same manner. The same method of administration was employed to effect oromucosal administration in the studies which are described below.

Six week old, male DBA/2 mice were treated with either 100,000 IU of recombinant murine interferon α (IFN α) purchased from Life Technologies Inc, in phosphate buffered saline (PBS), 10 μg of recombinant human interleukin 15 (IL-15) purchased from Protein Institute Inc, PBS containing 100 μg/ml of bovine serum albumin (BSA), or left untreated. Eight hours later, the mice were sacrificed by cervical dislocation and the lymphoid tissue was removed surgically from the oropharyngeal cavity and snap frozen in liquid nitrogen and stored at −80° C. RNA was extracted from the lymphoid tissue by the method of Chomczynski and Sacchi (Anal. Biochem. (1987) 162, 156-159) and subjected to. mRNA Differential Display Analysis (Lang, P. and Pardee, A. B., Science, 257, 967-971).

Differential Display Analysis

Differential display analysis was carried out using the “Message Clean” and “RNA image” kits of the GenHunter Corporation essentially as described by the manufacturer. Briefly, RNA was treated with RNase-free DNase, and 1 μg was reverse-transcribed in 100 μl of reaction buffer using either one or the other of the three one-base anchored oligo-(dT) primers A, C, or G. RNA was also reverse-transcribed using one or the other of the 9 two-base anchored oligo-(dT) primers AA, CC, GG, AC, CA, GA, AG, CG, GC. All the samples to be compared were reverse transcribed in the same experiment, separated into aliquots and frozen. The amplification was performed with only 1 μl of the reverse transcription sample in 10 μl of amplification mixture containing Taq DNA polymerase and α-³³P dATP (3,000 Ci/mmole). Eighty 5′ end (HAP) random sequence primers were used in combination with each of the (HT11) A, C, G, AA, CC, GG, AC, CA, GA, AG, CG or GC primers. Samples were then run on 7% denaturing polyacrylamide gels and exposed to authoradiography. Putative differentially expressed bands were cut out, reamplified according to the instructions of the supplier, and further used as probes to hybridise Northern blots of RNA extracted from the oropharyngeal cavity of IFN treated, IL-15 treated, and excipient treated animals.

Cloning and Sequencing

Re-amplified bands from the differential display screen were cloned in the SFr 1 site of the pPCR-Script SK(+) plasmid (Stratagene), and cDNA amplified from the rapid amplification of cDNA ends were isolated by TA cloning in the pCR3 plasmid (Invitrogen). DNA was sequenced using an automatic di-deoxy sequencer (Perkin Elmer ABI PRISM 377).

Identification of Human cDNA

Differentially expressed murine 3′ sequences identified from the differential display screen were compared with random human expressed sequence tags (EST) present in the dbEST database of GenBank™ of the United States National Center for Biotechnology Information (NCBI). The sequences potentially related to the murine EST isolated from the differential display screen were combined in a contig and used to construct a human consensus sequence corresponding to a putative cDNA.

One such cDNA was found to correspond to GenBank cDNA sequence g33917. The corresponding polypeptide sequence is GenBank sequence g33918, which is noted in GenBank as corresponding to IP-10.

Other mouse genes upregulated in lymphoid tissue in response to oromucosal administration of IFN-α as described above have also been found to be upregulated in the spleen of mice in response to intravenous administration of IFN-α. A similar result is anticipated in respect of the mouse gene corresponding to the human gene identified by Genbank cDNA accesssion no. g33917 when intravenous administration of IFN-α is carried out as described in Example 17 below.

Furthermore, mRNAs corresponding to human gene analogues of mouse genes found to be upregulated in response to oromucosal and intravenous administration of IFN-α have been found to be enhanced in human peripheral blood mononuclear cells following treatment with IFN-α in vitro. The same result is anticipated for mRNA corresponding to the cDNA as set forth in SEQ. ID. No. 9 when human peripheral blood mononuclear cells are treated with IFN-α as described in Example 18 below.

### Example 6

Previous experiments had shown that the application of 5 μl of crystal violet to each nostril of a normal adult mouse using a P20 Eppendorf micropipette resulted in an almost immediate distribution of the dye over the whole surface of the oropharyngeal cavity. Staining of the oropharyngeal cavity was still apparent some 30 minutes after application of the dye. These results were confirmed by using ¹²⁵I-labelled recombinant human IFN-α1-8 applied in the same manner. The same method of administration was employed to effect oromucosal administration in the studies which are described below.

Six week old, male DBA/2 mice were treated with either 100,000 IU of recombinant murine interferon α (IFN α) purchased from Life Technologies Inc, in phosphate buffered saline (PBS), 10 μg of recombinant human interleukin 15 (IL-15) purchased from Protein Institute Inc, PBS containing 100 μg/ml of bovine serum albumin (BSA), or left untreated. Eight hours later, the mice were sacrificed by cervical dislocation and the lymphoid tissue was removed surgically from the oropharyngeal cavity and snap frozen in liquid nitrogen and stored at −80° C. RNA was extracted from the lymphoid tissue by the method of Chomczynski and Sacchi (Anal. Biochem. (1987) 162, 156-159) and subjected to mRNA Differential Display Analysis (Lang, P. and Pardee, A. B., Science, 257, 967-971).

Differential Display Analysis

Differential display analysis was carried out using the “Message Clean” and “RNA image” kits of the GenHunter Corporation essentially as described by the manufacturer. Briefly, RNA was treated with RNase-free DNase, and 1 μg was reverse-transcribed in 100 μl of reaction buffer using either one or the other of the three one-base anchored oligo-(dT) primers A, C, or G. RNA was also reverse-transcribed using one or the other of the 9 two-base anchored oligo-(dT) primers AA, CC, GG, AC, CA, GA, AG, CG, GC. All the samples to be compared were reverse transcribed in the same experiment, separated into aliquots and frozen. The amplification was performed with only 1 μl of the reverse transcription sample in 10 μl of amplification mixture containing Taq DNA polymerase and α-33dATP (3,000 Ci/mmole). Eighty 5′ end (HAP) random sequence primers were used in combination with each of the (HT11) A, C, G, AA, CC, GG, AC, CA, GA, AG, CG or GC primers. Samples were then run on 7% denaturing polyacrylamide gels and exposed to authoradiography. Putative differentially expressed bands were cut out, reamplified according to the instructions of the supplier, and further used as probes to hybridise Northern blots of RNA extracted from the oropharyngeal cavity of IFN treated, IL-15 treated, and excipient treated animals.

Cloning and Sequencing

Re-amplified bands from the differential display screen were cloned in the Sfr 1 site of the pPCR-Script SK(+) plasmid (Stratagene), and cDNA amplified from the rapid amplification of cDNA ends were isolated by TA cloning in the pCR3 plasmid (Invitrogen). DNA was sequenced using an automatic di-deoxy sequencer (Perkin Elmer ABI PRISM 377).

Identification of Humnan cDNA

Differentially expressed murine 3′ sequences identified from the differential display screen were compared with random human expressed sequence tags (EST) present in the dbEST database of GenBank™ of the United States National Center for Biotechnology Information (NCBI). The sequences potentially related to the murine EST isolated from the differential display screen were combined in a contig and used to construct a human consensus sequence corresponding to a putative cDNA.

One such cDNA was found to correspond to GenBank cDNA sequence g4504962. The corresponding polypeptide sequence is GenBank sequence g4504963, which is noted in GenBank as corresponding to Lipocalin 1.

Other mouse genes upregulated in lymphoid tissue in response to oromucosal administration of IFN-α as described above have also been found to be upregulated in the spleen of mice in response to intravenous administration of IFN-α. A similar result is anticipated in respect of the mouse gene corresponding to the human gene identified by Genbank cDNA accesssion no. g4504962 when intravenous administration of IFN-α is carried out as described in Example 17 below.

Furthermore, mRNAs corresponding to human gene analogues of mouse genes found to be upregulated in response to oromucosal and intravenous administration of IFN-α have been found to be enhanced in human peripheral blood mononuclear cells following treatment with IFN-α in vitro. The same result is anticipated for mRNA corresponding to the cDNA as set forth in SEQ. ID. No. 11 when human peripheral blood mononuclear cells are treated with IFN-α as described in Example 18 below.

### Example 7

Previous experiments had shown that the application of 5 μl of crystal violet to each nostril of a normal adult mouse using a P20 Eppendorf micropipette resulted in an almost immediate distribution of the dye over the whole surface of the oropharyngeal cavity. Staining of the oropharyngeal cavity was still apparent some 30 minutes after application of the dye. These results were confirmed by using ¹²⁵I-labelled recombinant human IFN-α1-8 applied in the same manner. The same method of administration was employed to effect oromucosal administration in the studies which are described below.

Six week old, male DBA/2 mice were treated with either 100,000 IU of recombinant murine interferon α (IFN α) purchased from Life Technologies Inc, in phosphate buffered saline (PBS), 10 μg of recombinant human interleukin 15 (IL-15) purchased from Protein Institute Inc, PBS containing 100 μg/ml of bovine serum albumin (BSA), or left untreated. Eight hours later, the mice were sacrificed by cervical dislocation and the lymphoid tissue was removed surgically from the oropharyngeal cavity and snap frozen in liquid nitrogen and stored at −80° C. RNA was extracted from the lymphoid tissue by the method of Chomczynski and Sacchi (Anal. Biochem. (1987) 162, 156-159) and subjected to mRNA Differential Display Analysis (Lang, P. and Pardee, A. B., Science, 257, 967-971).

Differential Display Analysis

Differential display analysis was carried out using the “Message Clean” and “RNA image” kits of the GenHunter Corporation essentially as described by the manufacturer. Briefly, RNA was treated with RNase-free DNase, and 1 μg was reverse-transcribed in 100 μl of reaction buffer using either one or the other of the three one-base anchored oligo-(dT) primers A, C, or G. RNA was also reverse-transcribed using one or the other of the 9 two-base anchored oligo-(dT) primers AA, CC, GG, AC, CA, GA, AG, CG, GC. All the samples to be compared were reverse transcribed in the same experiment, separated into aliquots and frozen. The amplification was performed with only 1 μl of the reverse transcription sample in 10 μl of amplification mixture containing Taq DNA polymerase and α-³³P dATP (3,000 Ci/mmole). Eighty 5′ end (HAP) random sequence primers were used in combination with each of the (HT11) A, C, G, AA, CC, GG, AC, CA, GA, AG, CG or GC primers. Samples were then run on 7% denaturing polyacrylamide gels and exposed to authoradiography. Putative differentially expressed bands were cut out, reamplified according to the instructions of the supplier, and further used as probes to hybridise Northern blots of RNA extracted from the oropharyngeal cavity of IFN treated, IL-15 treated, and excipient treated animals.

Cloning and Sequencing

Re-amplified bands from the differential display screen were cloned in the Sfr 1 site of the pPCR-Script SK(+) plasmid (Stratagene), and cDNA amplified from the rapid amplification of cDNA ends were isolated by TA cloning in the pCR3 plasmid (Invitrogen). DNA was sequenced using an automatic di-deoxy sequencer (Perkin Elmer ABI PRISM 377).

Identification of Human cDNA

Differentially expressed murine 3′ sequences identified from the differential display screen were compared with random human expressed sequence tags (EST) present in the dbEST database of GenBank™ of the United States National Center for Biotechnology Information (NCBI). The sequences potentially related to the murine EST isolated from the differential display screen were combined in a contig and used to construct a human consensus sequence corresponding to a putative cDNA.

One such cDNA was found to correspond to GenBank cDNA sequence g3978516. The corresponding polypeptide sequence is GenBank sequence g3978517, which is noted in GenBank as corresponding to SEC 63.

Other mouse genes upregulated in lymphoid tissue in response to oromucosal administration of IFN-α as described above have also been found to be upregulated in the spleen of mice in response to intravenous administration of IFN-α. A similar result is anticipated in respect of the mouse gene corresponding to the human gene identified by Genbank cDNA accesssion no. g3978516 when intravenous administration of IFN-α is carried out as described in Example 17 below.

Furthermore, mRNAs corresponding to human gene analogues of mouse genes found to be upregulated in response to oromucosal and intravenous administration of IFN-α have been found to be enhanced in human peripheral blood mononuclear cells following treatment with IFN-α in vitro. The same result is anticipated for mRNA corresponding to the cDNA as set forth in SEQ. ID. No. 13 when human peripheral blood mononuclear cells are treated with IFN-α as described in Example 18 below.

### Example 8

Previous experiments had shown that the application of 5 μl of crystal violet to each nostril of a normal adult mouse using a P20 Eppendorf micropipette resulted in an almost immediate distribution of the dye over the whole surface of the oropharyngeal cavity. Staining of the oropharyngeal cavity was still apparent some 30 minutes after application of the dye. These results were confirmed by using ¹²⁵I-labelled recombinant human IFN-α1-8 applied in the same manner. The same method of administration was employed to effect oromucosal administration in the studies which are described below.

Six week old, male DBA/2 mice were treated with either 100,000 IU of recombinant murine interferon α (IFN α) purchased from Life Technologies Inc, in phosphate buffered saline (PBS), 10 μg of recombinant human interleukin 15 (L-15) purchased from Protein Institute Inc, PBS containing 100 μg/ml of bovine serum albumin (BSA), or left untreated. Eight hours later, the mice were sacrificed by cervical dislocation and the lymphoid tissue was removed surgically from the oropharyngeal cavity and snap frozen in liquid nitrogen and stored at −80° C. RNA was extracted from the lymphoid tissue by the method of Chomczynski and Sacchi (Anal. Biochem. (1987) 162, 156-159) and subjected to mRNA Differential Display Analysis (Lang, P. and Pardee, A. B., Science, 257, 967-971).

Differential Display Analysis

Differential display analysis was carried out using the “Message Clean” and “RNA image” kits of the GenHunter Corporation essentially as described by the manufacturer. Briefly, RNA was treated with RNase-free DNase, and 1 μg was reverse-transcribed in 100 μl of reaction buffer using either one or the other of the three one-base anchored oligo-(dT) primers A, C, or G. RNA was also reverse-transcribed using one or the other of the 9 two-base anchored oligo-(dT) primers AA, CC, GG, AC, CA, GA, AG, CG, GC. All the samples to be compared were reverse transcribed in the same experiment, separated into aliquots and frozen. The amplification was performed with only 1 μl of the reverse transcription sample in 10 μl of amplification mixture containing Taq DNA polymerase and α-³³P dATP (3,000 Ci/mmole). Eighty 5′ end (HAP) random sequence primers were used in combination with each of the (HT11) A, C, G, AA, CC, GG, AC, CA, GA, AG, CG or GC primers. Samples were then run on 7% denaturing polyacrylamide gels and exposed to authoradiography. Putative differentially expressed bands were cut out, reamplified according to the instructions of the supplier, and further used as probes to hybridise Northern blots of RNA extracted from the oropharyngeal cavity of IFN treated, IL-15 treated, and excipient treated animals.

Cloning and Sequencing

Re-amplified bands from the differential display screen were cloned in the Sfr 1 site of the pPCR-Script SK(+) plasmid (Stratagene), and cDNA amplified from the rapid amplification of cDNA ends were isolated by TA cloning in the pCR3 plasmid (Invitrogen). DNA was sequenced using an automatic di-deoxy sequencer (Perkin Elmer ABI PRISM 377).

Identification of Human cDNA

Differentially expressed murine 3′ sequences identified from the differential display screen were compared with random human expressed sequence tags (EST) present in the dbEST database of GenBank™ of the United States National Center for Biotechnology Information (NCBI). The sequences potentially related to the murine EST isolated from the differential display screen were combined in a contig and used to construct a human consensus sequence corresponding to a putative cDNA.

One such cDNA was found to correspond to GenBank cDNA sequence g5924396. The corresponding polypeptide sequence is GenBank sequence g5924397, which is noted in GenBank as corresponding to surfeit 6.

Other mouse genes upregulated in lymphoid tissue in response to oromucosal administration of IFN-α as described above have also been found to be upregulated in the spleen of mice in response to intravenous administration of IFN-α. A similar result is anticipated in respect of the mouse gene corresponding to the human gene identified by Genbank cDNA accesssion no. g5924396 when intravenous administration of IFN-α is carried out as described in Example 17 below.

Furthermore, mRNAs corresponding to human gene analogues of mouse genes found to be upregulated in response to oromucosal and intravenous administration of IFN-α have been found to be enhanced in human peripheral blood mononuclear cells following treatment with IFN-α in vitro. The same result is anticipated for mRNA corresponding to the cDNA as set forth in SEQ. ID. No. 15 when human peripheral blood mononuclear cells are treated with IFN-α as described in Example 18 below.

### Example 9

Previous experiments had shown that the application of 5 μl of crystal violet to each nostril of a normal adult mouse using a P20 Eppendorf micropipette resulted in an almost immediate distribution of the dye over the whole surface of the oropharyngeal cavity. Staining of the oropharyngeal cavity was still apparent some 30 minutes after application of the dye. These results were confirmed by using ¹²⁵I-labelled recombinant human IFN-α1-8 applied in the same manner. The same method of administration was employed to effect oromucosal administration in the studies which are described below.

Six week old, male DBA/2 mice were treated with either 100,000 IU of recombinant murine interferon α (IFN α) purchased from Life Technologies Inc, in phosphate buffered saline (PBS), 10 μg of recombinant human interleukin 15 (IL-15) purchased from Protein Institute Inc, PBS containing 100 μg/ml of bovine serum album (BSA), or left untreated. Eight hours later, the mice were sacrificed by cervical dislocation and the lymphoid tissue was removed surgically from the oropharyngeal cavity and snap frozen in liquid nitrogen and stored at −80° C. RNA was extracted from the lymphoid tissue by the method of Chomczynski and Sacchi (Anal. Biochem. (1987) 162, 156-159) and subjected to mRNA Differential Display Analysis (Lang, P. and Pardee, A. B., Science, 257, 967-971).

Differential Display Analysis

Differential display analysis was carried out using the “Message Clean” and “RNA image” kits of the GenHunter Corporation essentially as described by the manufacturer. Briefly, RNA was treated with RNase-free DNase, and 1 μg was reverse-transcribed in 100 μl of reaction buffer using either one or the other of the three one-base anchored oligo-(dT) primers A, C, or G. RNA was also reverse-transcribed using one or the other of the 9 two-base anchored oligo-(dT) primers AA, CC, GG, AC, CA, GA, AG, CG, GC. All the samples to be compared were reverse transcribed in the same experiment, separated into aliquots and frozen. The amplification was performed with only 1 μl of the reverse transcription sample in 10 μl of amplification mixture containing Taq DNA polymerase and α-³³P dATP (3,000 Ci/mmole). Eighty 5′ end (HAP) random sequence primers were used in combination with each of the (HT11) A, C, G, AA, CC, GG, AC, CA, GA, AG, CG or GC primers. Samples were then run on 7% denaturing polyacrylamide gels and exposed to authoradiography. Putative differentially expressed bands were cut out, reamplified according to the instructions of the supplier, and further used as probes to hybridise Northern blots of RNA extracted from the oropharyngeal cavity of IFN treated, IL-15 treated, and excipient treated animals.

Cloning and Sequencing

Re-amplified bands from the differential display screen were cloned in the Sfr 1 site of the pPCR-Script SK(+) plasmid (Stratagene), and cDNA amplified from the rapid amplification of cDNA ends were isolated by TA cloning in the pCR3 plasmid (Invitrogen). DNA was sequenced using an automatic di-deoxy sequencer (Perkin Elmer ABI PRISM 377).

Identification of Human cDNA

Differentially expressed murine 3′ sequences identified from the differential display screen were compared with random human expressed sequence tags (EST) present in the dbEST database of GenBank™ of the United States National Center for Biotechnology Information (NCBI). The sequences potentially related to the murine EST isolated from the differential display screen were combined in a contig and used to construct a human consensus sequence corresponding to a putative cDNA.

One such cDNA was found to correspond to GenBank cDNA sequence g4505656. The corresponding polypeptide sequence is GenBank sequence g4505657, which is noted in GenBank as corresponding to PDE2A.

Other mouse genes upregulated in lymphoid tissue in response to oromucosal administration of IFN-α as described above have also been found to be upregulated in the spleen of mice in response to intravenous administration of IFN-α. A similar result is anticipated in respect of the mouse gene corresponding to the human gene identified by Genbank cDNA accesssion no. g4505656 when intravenous administration of IFN-α is carried out as described in Example 17 below.

Furthermore, mRNAs corresponding to human gene analogues of mouse genes found to be upregulated in response to oromucosal and intravenous administration of IFN-α have been found to be enhanced in human peripheral blood mononuclear cells following treatment with IFN-α in vitro. The same result is anticipated for mRNA corresponding to the cDNA as set forth in SEQ. ID. No. 17 when human peripheral blood mononuclear cells are treated with IFN-α as described in Example 18 below.

### Example 10

Previous experiments had shown that the application of 5 μl of crystal violet to each nostril of a normal adult mouse using a P20 Eppendorf micropipette resulted in an almost immediate distribution of the dye over the whole surface of the oropharyngeal cavity. Staining of the oropharyngeal cavity was still apparent some 30 minutes after application of the dye. These results were confirmed by using ¹²⁵I-labelled recombinant human IFN-α1-8 applied in the same manner. The same method of administration was employed to effect oromucosal administration in the studies which are described below.

Six week old, male DBA/2 mice were treated with either 100,000 IU of recombinant murine interferon α (IFN α) purchased from Life Technologies Inc, in phosphate buffered saline (PBS), 10 μg of recombinant human interleukin 15 (IL-15) purchased from Protein Institute Inc, PBS containing 100 μg/ml of bovine serum albumin (BSA), or left untreated. Eight hours later, the mice were sacrificed by cervical dislocation and the lymphoid tissue was removed surgically from the oropharyngeal cavity and snap frozen in liquid nitrogen and stored at −80° C. RNA was extracted from the lymphoid tissue by the method of Chomczynski and Sacchi (Anal. Biochem. (1987) 162, 156-159) and subjected to mRNA Differential Display Analysis (Lang, P. and Pardee, A.B., Science, 257, 967-971).

Differential Display Analysis

Differential display analysis was carried out using the “Message Clean” and “RNA image” kits of the GenHunter Corporation essentially as described by the manufacturer. Briefly, RNA was treated with RNase-free DNase, and 1 μg was reverse-transcribed in 100 μl of reaction buffer using either one or the other of the three one-base anchored oligo-(dT) primers A, C, or G. RNA was also reverse-transcribed using one or the other of the 9 two-base anchored oligo-(dT) primers AA, CC, GG, AC, CA, GA, AG, CG, GC. All the samples to be compared were reverse transcribed in the same experiment, separated into aliquots and frozen. The amplification was performed with only 1 μl of the reverse transcription sample in 10 μl of amplification mixture containing Taq DNA polymerase and α-³³P dATP (3,000 Ci/mmole). Eighty 5′ end (HAP) random sequence primers were used in combination with each of the (HT11) A, C, G, AA, CC, GG, AC, CA, GA, AG, CG or GC primers. Samples were then run on 7% denaturing polyacrylamide gels and exposed to authoradiography. Putative differentially expressed bands were cut out, reamplified according to the instructions of the supplier, and further used as probes to hybridise Northern blots of RNA extracted from the oropharyngeal cavity of IFN treated, IL-15 treated, and excipient treated animals.

Cloning and Sequencing

Re-amplified bands from the differential display screen were cloned in the Sfr 1 site of the pPCR-Script SK(+) plasmid (Stratagene), and cDNA amplified from the rapid amplification of cDNA ends were isolated by TA cloning in the pCR3 plasmid (Invitrogen). DNA was sequenced using an automatic di-deoxy sequencer (Perkin Elmer ABI PRISM 377).

Identification of Human cDNA

Differentially expressed murine 3′ sequences identified from the differential display screen were compared with random human expressed sequence tags (EST) present in the dbEST database of GenBank™ of the United States National Center for Biotechnology Information (NCBI). The sequences potentially related to the murine EST isolated from the differential display screen were combined in a contig and used to construct a human consensus sequence corresponding to a putative cDNA.

One such cDNA was found to correspond to GenBank cDNA sequence g1504007. The corresponding polypeptide sequence is GenBank sequence g1504008, which is noted in GenBank as corresponding to KIAA0212.

Other mouse genes upregulated in lymphoid tissue in response to oromucosal administration of IFN-α as described above have also been found to be upregulated in the spleen of mice in response to intravenous administration of IFN-α. A similar result is anticipated in respect of the mouse gene corresponding to the human gene identified by Genbank cDNA accesssion no. g1504007 when intravenous administration of IFN-α is carried out as described in Example 17 below.

Furthermore, mRNAs corresponding to human gene analogues of mouse genes found to be upregulated in response to oromucosal and intravenous administration of IFN-α have been found to be enhanced in human peripheral blood mononuclear cells following treatment with IFN-α in vitro. The same result is anticipated for mRNA corresponding to the cDNA as set forth in SEQ. ID. No. 119 when human peripheral blood mononuclear cells are treated with IFN-α as described in Example 18 below.

### Example 11

Previous experiments had shown that the application of 5 μl of crystal violet to each nostril of a normal adult mouse using a P20 Eppendorf micropipette resulted in an almost immediate distribution of the dye over the whole surface of the oropharyngeal cavity. Staining of the oropharyngeal cavity was still apparent some 30 minutes after application of the dye. These results were confirmed by using ¹²⁵I-labelled recombinant human IFN-α1-8 applied in the same manner. The same method of administration was employed to effect oromucosal administration in the studies which are described below.

Six week old, male DBA/2 mice were treated with either 100,000 IU of recombinant murine interferon α (IFN α) purchased from Life Technologies Inc, in phosphate buffered saline (PBS), 10 μg of recombinant human interleukin 15 (IL-15) purchased from Protein Institute Inc, PBS containing 100 μg/ml of bovine serum albumin (BSA), or left untreated. Eight hours later, the mice were sacrificed by cervical dislocation and the lymphoid tissue was removed surgically from the oropharyngeal cavity and snap frozen in liquid nitrogen and stored at −80° C. RNA was extracted from the lymphoid tissue by the method of Chomczynski and Sacchi (Anal. Biochem. (1987) 162, 156-159) and subjected to mRNA Differential Display Analysis (Lang, P. and Pardee, A. B., Science, 257, 967-971).

Differential Display Analysis

Differential display analysis was carried out using the “Message Clean” and “RNA image” kits of the GenHunter Corporation essentially as described by the manufacturer. Briefly, RNA was treated with RNase-free DNase, and 1 μg was reverse-transcribed in 100 μl of reaction buffer using either one or the other of the three one-base anchored oligo-(dT) primers A, C, or G. RNA was also reverse-transcribed using one or the other of the 9 two-base anchored oligo-(dT) primers AA, CC, GG, AC, CA, GA, AG, CG, GC. All the samples to be compared were reverse transcribed in the same experiment, separated into aliquots and frozen. The amplification was performed with only 1 μl of the reverse transcription sample in 10 μl of amplification mixture containing Taq DNA polymerase and α-³³P dATP (3,000 Ci/mmole). Eighty 5′ end (HAP) random sequence primers were used in combination with each of the (HT11) A, C, G, AA, CC, GG, AC, CA, GA, AG, CG or GC primers. Samples were then run on 7% denaturing polyacrylamide gels and exposed to authoradiography. Putative differentially expressed bands were cut out, reamplified according to the instructions of the supplier, and further used as probes to hybridise Northern blots of RNA extracted from the oropharyngeal cavity of IFN treated, IL-15 treated, and excipient treated animals.

Cloning and Sequencing

Re-amplified bands from the differential display screen were cloned in the Sfr 1 site of the pPCR-Script SK(+) plasmid (Stratagene), and cDNA amplified from the rapid amplification of cDNA ends were isolated by TA cloning in the pCR3 plasmid (Invitrogen). DNA was sequenced using an automatic di-deoxy sequencer (Perkin Elmer ABI PRISM 377).

Identification of Human cDNA

Differentially expressed murine 3′ sequences identified from the differential display screen were compared with random human expressed sequence tags (EST) present in the dbEST database of GenBank™ of the United States National Center for Biotechnology Information (NCBI). The sequences potentially related to the murine EST isolated from the differential display screen were combined in a contig and used to construct a human consensus sequence corresponding to a putative cDNA.

One such cDNA was found to correspond to GenBank cDNA sequence g3702446. The corresponding polypeptide sequence is GenBank sequence g3702448, which is noted in GenBank as corresponding to. NPIK-B.

Other mouse genes upregulated in lymphoid tissue in response to oromucosal administration of IFN-α as described above have also been found to be upregulated in the spleen of mice in response to intravenous administration of IFN-α. A similar result is anticipated in respect of the mouse gene corresponding to the human gene identified by Genbank eDNA accesssion no. g3702446 when intravenous administration of IFN-α is carried out as described in Example 17 below.

Furthermore, mRNAs corresponding to human gene analogues of mouse genes found to be upregulated in response to oromucosal and intravenous administration of IFN-α have been found to be enhanced in human peripheral blood mononuclear cells following treatment with IFN-α in vitro. The same result is anticipated for mRNA corresponding to the cDNA as set forth in SEQ. ID. No. 21 when human peripheral blood mononuclear cells are treated with IFN-α as described in Example 18 below.

### Example 12

Previous experiments had shown that the application of 5 μl of crystal violet to each nostril of a normal adult mouse using a P20 Eppendorf micropipette resulted in an almost immediate distribution of the dye over the whole surface of the oropharyngeal cavity. Staining of the oropharyngeal cavity was still apparent some 30 minutes after application of the dye. These results were confirmed by using ¹²⁵I-labelled recombinant human IFN-α1-8 applied in the same manner. The same method of administration was employed to effect oromucosal administration in the studies which are described below.

Six week old, male DBA/2 mice were treated with either 100,000 IU of recombinant murine interferon α (IFN α) purchased from Life Technologies Inc, in phosphate buffered saline (PBS), 10 μg of recombinant human interleukin 15 (IL-15) purchased from Protein Institute Inc, PBS containing 100 μg/ml of bovine serum albumin (BSA), or left untreated. Eight hours later, the mice were sacrificed by cervical dislocation and the lymphoid tissue was removed surgically from the oropharyngeal cavity and snap frozen in liquid nitrogen and stored at −80° C. RNA was extracted from the lymphoid tissue by the method of Chomczynski and Sacchi (Anal. Biochem. (1987) 162, 156-159) and subjected to mRNA Differential Display Analysis (Lang, P. and Pardee, A. B., Science, 257, 967-971).

Differential Display Analysis

Differential display analysis was carried out using the “Message Clean” and “RNA image” kits of the GenHunter Corporation essentially as described by the manufacturer. Briefly, RNA was treated with RNase-free DNase, and 1 μg was reverse-transcribed in 100 μl of reaction buffer using either one or the other of the three one-base anchored oligo-(dT) primers A, C, or G. RNA was also reverse-transcribed using one or the other of the 9 two-base anchored oligo-(dT) primers AA, CC, GG, AC, CA, GA, AG, CG, GC. All the samples to be compared were reverse transcribed in the same experiment, separated into aliquots and frozen. The amplification was performed with only 1 μl of the reverse transcription sample in 10 μl of amplification mixture containing Taq DNA polymerase and α-³³P dATP (3,000 Ci/mmole). Eighty 5′ end (HAP) random sequence primers were used in combination with each of the (HT11) A, C, G, AA, CC, GG, AC, CA, GA, AG, CG or GC primers. Samples were then run on 7% denaturing polyacrylamide gels and exposed to authoradiography. Putative differentially expressed bands were cut out, reamplified according to the instructions of the supplier, and further used as probes to hybridise Northern blots of RNA extracted from the oropharyngeal cavity of IFN treated, IL-15 treated, and excipient treated animals.

Cloning and Sequencing

Re-amplified bands from the differential display screen were cloned in the Sfr 1 site of the pPCR-Script SK(+) plasmid (Stratagene), and cDNA amplified from the rapid amplification of cDNA ends were isolated by TA cloning in the pCR3 plasmid (Invitrogen). DNA was sequenced using an automatic di-deoxy sequencer (Perkin Elmer ABI PRISM 377).

Identification of Human cDNA

Differentially expressed murine 3′ sequences identified from the differential display screen were compared with random human expressed sequence tags (EST) present in the dbEST database of GenBank™ of the United States National Center for Biotechnology Information (NCBI). The sequences potentially related to the murine EST isolated from the differential display screen were combined in a contig and used to construct a human consensus sequence corresponding to a putative cDNA.

One such cDNA was found to correspond to GenBank cDNA sequence g4001802. The corresponding polypeptide sequence is GenBank sequence g4001803, which is noted in GenBank as corresponding to BAF53a.

Other mouse genes upregulated in lymphoid tissue in response to oromucosal administration of IFN-α as described above have also been found to be upregulated in the spleen of mice in response to intravenous administration of IFN-α. A similar result is anticipated in respect of the mouse gene corresponding to the human gene identified by Genbank cDNA accesssion no. g4001802 when intravenous administration of IFN-α is carried out as described in Example 17 below.

Furthermore, mRNAs corresponding to human gene analogues of mouse genes found to be upregulated in response to oromucosal and intravenous administration of IFN-α have been found to be enhanced in human peripheral blood mononuclear cells following treatment with IFN-α in vitro. The same result is anticipated for mRNA corresponding to the cDNA as set forth in SEQ. ID. No. 23 when human peripheral blood mononuclear cells are treated with IFN-α as described in Example 18 below.

### Example 13

Previous experiments had shown that the application of 5 μl of crystal violet to each nostril of a normal adult mouse using a P20 Eppendorf micropipette resulted in an almost immediate distribution of the dye over the whole surface of the oropharyngeal cavity. Staining of the oropharyngeal cavity was still apparent some 30 minutes after application of the dye. These results were confirmed by using ¹²⁵I-labelled recombinant human IFN-α1-8 applied in the same manner. The same method of administration was employed to effect oromucosal administration in the studies which are described below.

Six week old, male DBA/2 mice were treated with either 100,000 IU of recombinant murine interferon α (IFN α) purchased from Life Technologies Inc, in phosphate buffered saline (PBS), 10 μg of recombinant human interleukin 15 (IL-15) purchased from Protein Institute Inc, PBS containing 100 μg/ml of bovine serum albumin (BSA), or left untreated. Eight hours later, the mice were sacrificed by cervical dislocation and the lymphoid tissue was removed surgically from the oropharyngeal cavity and snap frozen in liquid nitrogen and stored at −80° C. RNA was extracted from the lymphoid tissue by the method of Chomczynski and Sacchi (Anal. Biochem. (1987) 162, 156-159) and subjected to mRNA Differential Display Analysis (Lang, P. and Pardee, A. B., Science, 257, 967-971).

Differential Display Analysis

Differential display analysis was carried out using the “Message Clean” and “RNA image” kits of the GenHunter Corporation essentially as described by the manufacturer. Briefly, RNA was treated with RNase-free DNase, and 1 μg was reverse-transcribed in 100 μl of reaction buffer using either one or the other of the three one-base anchored oligo-(dT) primers A, C, or G. RNA was also reverse-transcribed using one or the other of the 9 two-base anchored oligo-(dT) primers AA, CC, GG, AC, CA, GA, AG, CG, GC. All the samples to be compared were reverse transcribed in the same experiment, separated into aliquots and frozen. The amplification was performed with only 1 μl of the reverse transcription sample in 10 μl of amplification mixture containing Taq DNA polymerase and α-³³P dATP (3,000 Ci/mmole). Eighty 5′ end (HAP) random sequence primers were used in combination with each of the (HT11) A, C, G, AA, CC, GG, AC, CA, GA, AG, CG or GC primers. Samples were then run on 7% denaturing polyacrylamide gels and exposed to authoradiography. Putative differentially expressed bands were cut out, reamplified according to the instructions of the supplier, and further used as probes to hybridise Northern blots of RNA extracted from the oropharyngeal cavity of IFN treated, IL-15 treated, and excipient treated animals.

Cloning and Sequencing

Re-amplified bands from the differential display screen were cloned in the Sfr 1 site of the pPCR-Script SK(+) plasmid (Stratagene), and cDNA amplified from the rapid amplification of cDNA ends were isolated by TA cloning in the pCR3 plasmid (Invitrogen). DNA was sequenced using an automatic di-deoxy sequencer (Perkin Elmer ABI PRISM 377).

Identification of Human cDNA

Differentially expressed murine 3′ sequences identified from the differential display screen were compared with random human expressed sequence tags (EST) present in the dbEST database of GenBank™ of the United States National Center for Biotechnology Information (NCBI). The sequences potentially related to the murine EST isolated from the differential display screen were combined in a contig and used to construct a human consensus sequence corresponding to a putative cDNA.

One such cDNA was found to correspond to GenBank cDNA sequence g292289. The corresponding polypeptide sequence is GenBank sequence g292290, which is noted in GenBank as corresponding to MEF2C.

Other mouse genes upregulated in lymphoid tissue in response to oromucosal administration of IFN-α as described above have also been found to be upregulated in the spleen of mice in response to intravenous administration of IFN-α. A similar result is anticipated in respect of the mouse gene corresponding to the human gene identified by Genbank cDNA accesssion no. g292289 when intravenous administration of IFN-α is carried out as described in Example 17 below.

Furthermore, mRNAs corresponding to human gene analogues of mouse genes found to be upregulated in response to oromucosal and intravenous administration of IFN-α have been found to be enhanced in human peripheral blood mononuclear cells following treatment with IFN-α in vitro. The same result is anticipated for mRNA corresponding to the cDNA as set forth in SEQ. ID. No. 25 when human peripheral blood mononuclear cells are treated with IFN-α as described in Example 18 below.

### Example 14

Previous experiments had shown that the application of 5 μl of crystal violet to each nostril of a normal adult mouse using a P20 Eppendorf micropipette resulted in an almost immediate distribution of the dye over the whole surface of the oropharyngeal cavity. Staining of the oropharyngeal cavity was still apparent some 30 minutes after application of the dye. These results were confirmed by using ¹²⁵I-labelled recombinant human IFN-α1-8 applied in the same manner. The same method of administration was employed to effect oromucosal administration. in the studies which are described below.

Six week old, male DBA/2 mice were treated with either 100,000 IU of recombinant murine interferon α (IFN α) purchased from Life Technologies Inc, in phosphate buffered saline (PBS), 10 μg of recombinant human interleukin 15 (IL-15) purchased from Protein Institute Inc, PBS containing 100 μg/ml of bovine serum albumin (BSA), or left untreated. Eight hours later, the mice were sacrificed by cervical dislocation and the lymphoid tissue was removed surgically from the oropharyngeal cavity and snap frozen in liquid nitrogen and stored at −80° C. RNA was extracted from the lymphoid tissue by the method of Chomczynski and Sacchi (Anal. Biochem. (1987) 162, 156-159) and subjected to mRNA Differential Display Analysis (Lang, P. and Pardee, A.B., Science, 257, 967-971).

Differential Display Analysis

Differential display analysis was carried out using the “Message Clean” and “RNA image” kits of the GenHunter Corporation essentially as described by the manufacturer. Briefly, RNA was treated with RNase-free DNase, and 1 μg was reverse-transcribed in 100 μl of reaction buffer using either one or the other of the three one-base anchored oligo-(dT) primers A, C, or G. RNA was also reverse-transcribed using one or the other of the 9 two-base anchored oligo-(dT) primers AA, CC, GG, AC, CA, GA, AG, CG, GC. All the samples to be compared were reverse transcribed in the same experiment, separated into aliquots and frozen. The amplification was performed with only 1 μl of the reverse transcription sample in 10 μl of amplification mixture containing Taq DNA polymerase and α-³³P dATP (3,000 Ci/mmole). Eighty 5′ end (HAP) random sequence primers were used in combination with each of the (HT11) A, C, G, AA, CC, GG, AC, CA, GA, AG, CG or GC primers. Samples were then run on 7% denaturing polyacrylamide gels and exposed to authoradiography. Putative differentially expressed bands were cut out, reamplified according to the instructions of the supplier, and further used as probes to hybridise Northern blots of RNA extracted from the oropharyngeal cavity of IFN treated, IL-15 treated, and excipient treated animals.

Cloning and Sequencing

Re-amplified bands from the differential display screen were cloned in the Sfr I site of the pPCR-Script SK(+) plasmid (Stratagene), and cDNA amplified from the rapid amplification of cDNA ends were isolated by TA cloning in the pCR3 plasmid (Invitrogen). DNA was sequenced using an automatic di-deoxy sequencer (Perkin Elmer ABI PRISM 377).

Identification of Human cDNA

Differentially expressed murine 3′ sequences identified from the differential display screen were compared with random human expressed sequence tags (EST) present in the dbEST database of GenBank™ of the United States National Center for Biotechnology Information (NCBI). The sequences potentially related to the murine EST isolated from the differential display screen were combined in a contig and used to construct a human consensus sequence corresponding to a putative cDNA.

One such cDNA was found to correspond to GenBank cDNA sequence g4557226. The corresponding polypeptide sequence is GenBank sequence g4557227, which is noted in GenBank as corresponding to AADAC.

Other mouse genes upregulated in lymphoid tissue in response to oromucosal administration of IFN-α as described above have also been found to be upregulated in the spleen of mice in response to intravenous administration of IFN-α. A similar result is anticipated in respect of the mouse gene corresponding to the human gene identified by Genbank cDNA accesssion no. g4557226 when intravenous administration of IFN-α is carried out as described in Example 17 below.

Furthermore, mRNAs corresponding to human gene analogues of mouse genes found to be upregulated in response to oromucosal and intravenous administration of IFN-α have been found to be enhanced in human peripheral blood mononuclear cells following treatment with IFN-α in vitro. The same result is anticipated for mRNA corresponding to the cDNA as set forth in SEQ. ID. No. 27 when human peripheral blood mononuclear cells are treated with IFN-α as described in Example 18 below.

### Example 15

Previous experiments had shown that the application of 5 μl of crystal violet to each nostril of a normal adult mouse using a P20 Eppendorf micropipette resulted in an almost immediate distribution of the dye over the whole surface of the oropharyngeal cavity. Staining of the oropharyngeal cavity was still apparent some 30 minutes after application of the dye. These results were confirmed by using ¹²⁵I-labelled recombinant human IFN-α1-8 applied in the same manner. The same method of administration was employed to effect oromucosal administration in the studies which are described below.

Six week old, male DBA/2 mice were treated with either 100,000 IU of recombinant murine interferon α (IFN α) purchased from Life Technologies Inc, in phosphate buffered saline (PBS), 10 μg of recombinant human interleukin 15 (IL-15) purchased from Protein Institute Inc, PBS containing 100 μg/ml of bovine serum albumin (BSA), or left untreated. Eight hours later, the mice were sacrificed by cervical dislocation and the lymphoid tissue was removed surgically from the oropharyngeal cavity and snap frozen in liquid nitrogen and stored at −80° C. RNA was extracted from the lymphoid tissue by the method of Chomczynski and Sacchi (Anal. Biochem. (1987) 162, 156-159) and subjected to mRNA Differential Display Analysis (Lang, P. and Pardee, A. B., Science, 257, 967-971).

Differential Display Analysis

Differential display analysis was carried out using the “Message Clean” and “RNA image” kits of the GenHunter Corporation essentially as described by the manufacturer. Briefly, RNA was treated with RNase-free DNase, and 1 μg was reverse-transcribed in 100 μl of reaction buffer using either one or the other of the three one-base anchored oligo-(dT) primers A, C, or G. RNA was also reverse-transcribed using one or the other of the. 9 two-base anchored oligo-(dT) primers AA, CC, GG, AC, CA, GA, AG, CG, GC. All the samples to be compared were reverse transcribed in the same experiment, separated into aliquots and frozen. The amplification was performed with only 1 μl of the reverse transcription sample in 10 μl of amplification mixture containing Taq DNA polymerase and α-³³P dATP (3,000 Ci/mmole). Eighty 5′ end (HAP) random sequence primers were used in combination with each of the (HT11) A, C, G, AA, CC, GG, AC, CA, GA, AG, CG or GC primers. Samples were then run on 7% denaturing polyacrylamide gels and exposed to authoradiography. Putative differentially expressed bands were cut out, reamplified according to the instructions of the supplier, and further used as probes to hybridise Northern blots of RNA extracted from the oropharyngeal cavity of IFN treated, IL-15 treated, and excipient treated animals.

Cloning and Sequencing

Re-amplified bands from the differential display screen were cloned in the Sfr 1 site of the pPCR-Script SK(+) plasmid (Stratagene), and cDNA amplified from the rapid amplification of cDNA ends were isolated by TA cloning in the pCR3 plasmid (Invitrogen). DNA was sequenced using an automatic di-deoxy sequencer (Perkin Elmer ABI PRISM 377).

Identification of Human cDNA

Differentially expressed murine 3′ sequences identified from the differential display screen were compared with random human expressed sequence tags (EST) present in the dbEST database of GenBank™ of the United States National Center for Biotechnology Information (NCBI). The sequences potentially related to the murine EST isolated from the differential display screen were combined in a contig and used to construct a human consensus sequence corresponding to a putative cDNA.

One such cDNA was found to correspond to GenBank cDNA sequence g4507646. The corresponding polypeptide sequence is GenBank sequence g4507647, which is noted in GenBank as corresponding to TPM1.

Other mouse genes upregulated in lymphoid tissue in response to oromucosal administration of IFN-α as described above have also been found to be upregulated in the spleen of mice in response to intravenous administration of IFN-α. A similar result is anticipated in respect of the mouse gene corresponding to the human gene identified by Genbank eDNA accesssion no. g4507646 when intravenous administration of IFN-α is carried out as described in Example 17 below.

Furthermore, mRNAs corresponding to human gene analogues of mouse genes found to be upregulated in response to oromucosal and intravenous administration of IFN-α have been found to be enhanced in human peripheral blood mononuclear cells following treatment with IFN-α in vitro. The same result is anticipated for mRNA corresponding to the cDNA as set forth in SEQ. ID. No. 29 when human peripheral blood mononuclear cells are treated with IFN-α as described in Example 18 below.

### Example 16

Previous experiments had shown that the application of 5 μl of crystal violet to each nostril of a normal adult mouse using a P20 Eppendorf micropipette resulted in an almost immediate distribution of the dye over the whole surface of the oropharyngeal cavity. Staining of the oropharyngeal cavity was still apparent some 30 minutes after application of the dye. These results were confirmed by using ¹²⁵I-labelled recombinant human IFN-α1-8 applied in the same manner. The same method of administration was employed to effect oromucosal administration in the studies which are described below.

Six week old, male DBA/2 mice were treated with either 100,000 IU of recombinant murine interferon α (IFN α) purchased from Life Technologies Inc, in phosphate buffered saline (PBS), 10 μg of recombinant human interleukin 15 (IL-15) purchased from Protein Institute Inc, PBS containing 100 μg/ml of bovine serum albumin (BSA), or left untreated. Eight hours later, the mice were sacrificed by cervical dislocation and the lymphoid tissue was removed surgically from the oropharyngeal cavity and snap frozen in liquid nitrogen and stored at −80° C. RNA was extracted from the lymphoid tissue by the method of Chomczynski and Sacchi (Anal. Biochem. (1987) 162, 156-159) and subjected to mRNA Differential Display Analysis (Lang, P. and Pardee, A.B., Science, 257, 967-971).

Differential Display Analysis

Differential display analysis was carried out using the “Message Clean” and “RNA image” kits of the GenHunter Corporation essentially as described by the manufacturer. Briefly, RNA was treated with RNase-free DNase, and 1 μg was reverse-transcribed in 100 μl of reaction buffer using either one or the other of the three one-base anchored oligo-(dT) primers A, C, or G. RNA was also reverse-transcribed using one or the other of the 9 two-base anchored oligo-(dT) primers AA, CC, GG, AC, CA, GA, AG, CG, GC. All the samples to be compared were reverse transcribed in the same experiment, separated into aliquots and frozen. The amplification was performed with only 1 μl of the reverse transcription sample in 10 μl of amplification mixture containing Taq DNA polymerase and α-³³P dATP (3,000 Ci/mmole). Eighty 5′ end (HAP) random sequence primers were used in combination with each of the (HT11) A, C, G, AA, CC, GG, AC, CA, GA, AG, CG or GC primers. Samples were then run on 7% denaturing polyacrylamide gels and exposed to authoradiography. Putative differentially expressed bands were cut out, reamplified according to the instructions of the supplier, and further used as probes to hybridise Northern blots of RNA extracted from the oropharyngeal cavity of IFN treated, IL-15 treated, and excipient treated animals.

Cloning and Sequencing

Re-amplified bands from the differential display screen were cloned in the Sfr 1 site of the pPCR-Script SK(+) plasmid (Stratagene), and cDNA amplified from the rapid amplification of cDNA ends were isolated by TA cloning in the pCR3 plasmid (Invitrogen). DNA was sequenced using an automatic di-deoxy sequencer (Perkin Elmer ABI PRISM 377).

Identification of Human cDNA

Differentially expressed murine 3′ sequences identified from the differential display screen were compared with random human expressed sequence tags (EST) present in the dbEST database of GenBank™ of the United States National Center for Biotechnology Information (NCBI). The sequences potentially related to the murine EST isolated from the differential display screen were combined in a contig and used to construct a human consensus sequence corresponding to a putative cDNA.

One such cDNA was found to correspond to GenBank cDNA sequence g4507170. The corresponding polypeptide sequence is GenBank sequence g4507171, which is noted in GenBank as corresponding to SPARC.

Other mouse genes upregulated in lymphoid tissue in response to oromucosal administration of IFN-α as described above have also been found to be upregulated in the spleen of mice in response to intravenous administration of IFN-α. A similar result is anticipated in respect of the mouse gene corresponding to the human gene identified by Genbank cDNA accession no. g4507170 when intravenous administration of IFN-α is carried out as described in Example 17 below.

Furthermore, mRNAs corresponding to human gene analogues of mouse genes found to be upregulated in response to oromucosal and intravenous administration of IFN-α have been found to be enhanced in human peripheral blood mononuclear cells following treatment with IFN-α in vitro. The same result is anticipated for mRNA corresponding to the cDNA as set forth in SEQ. ID. No. 31 when human peripheral blood mononuclear cells are treated with IFN-α as described in Example 18 below.

### Example 17

Intravenous Administration of IFN-α

Male DBA/2 mice are injected intravenously with 100,000 IU of recombinant murine IFN-α purchased from Life Technologies Inc. in 200 μl of PBS or treated with an equal volume of PBS alone. Eight hours later the animals are sacrificed by cervical dislocation and the spleen was removed using conventional procedures. Total RNA was extracted by the method of Chomczynski and Sacchi (Anal. Biochem. (1987) 162, 156-159) and 10.0 μg of total RNA per sample is subjected to Northern blotting in the presence of glyoxal and hybridised with a cDNA probe for the mRNA of interest as described by Dandoy-Dron et al. (J. Biol. Chem. (1998) 273, 7691-7697). The blots are first exposed to autoradiography and then quantified using a Phospholmager according to the manufacturer's instructions.

### Example 18

Testing Type 1 Interferon Responsiveness in vitro

Human peripheral blood mononuclear cells (PBMC) from normal donors are isolated on Ficoll-Hypaque density gradients and treated in vitro with 10,000 IU of recombinant human IFN-α2 (intron A from Schering-Plough) in PBS or with an equal volume of PBS alone. Eight hours later the cells are centrifuged (800×g for 10 minutes) and the cell pellet recovered. Total RNA is extracted from the cell pellet by the method of Chomczynski and Sacchi and 10.0 μg of total RNA per sample is subjected to Northern blotting as described in Example 17 above.

The same procedure can be used to predict Type 1 interferon responsiveness using PBMC taken from a patient proposed to be treated with a Type 1 interferon.

1. A method of predicting responsiveness of a patient to treatment with a Type 1 interferon, which comprises determining the level of one or more proteins selected from the proteins defined by the sequences set forth in SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30 or SEQ ID NO: 32 and naturally-occurring variants thereof, or one or more of the corresponding mRNAs, in a cell sample from said patient, wherein said sample is obtained from said patient following administration of a Type 1 interferon or is treated prior to said determining with a Type 1 interferon in vitro.

2. A method as claimed in claim 1 wherein the interferon administered prior to obtaining said sample or used to treat said sample in vitro is the interferon proposed for treatment of the patient.

3. A method as claimed in claim 1 or 2 wherein a sample comprising peripheral blood mononuclear cells isolated from a blood sample of the patient is treated with a Type 1 interferon in vitro.

4. A method as claimed in any one of claims 1 to 3 wherein said determining comprises determining the level of one or more mRNAs encoding a protein selected from the proteins defined by the sequences set forth in SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30 or SEQ ID NO: 32 and naturally-occurring variants of said proteins.

5. A method as claimed in claim 4 wherein said mRNA, or a portion thereof, is amplified prior to detection.

6. A method as claimed in claim 4 or claim 5 wherein said mRNA, or an amplification product thereof, is detected by using a nucleic acid probe attached to a solid support.

7. A method as claimed in any one of claims 1 to 3 wherein said determining comprises determining the level of one or more proteins selected from the proteins defined by the sequences set forth in SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22,
SEQ ID NO: 24, SEQ ID NO: 26, SEQ ID NO: 28, SEQ ID NO: 30 or SEQ ID NO: 32 and naturally-occurring variants thereof.
